SEQUENCE LISTING (101 sequences)

SEQ ID NO: 1 - protein, 15 aa, Artificial Sequence
  Description of Artificial Sequence Synthetic 75-12 peptide
PETTDLYSYEQLNDS

SEQ ID NO: 2 - protein, 15 aa, Artificial Sequence
  Description of Artificial Sequence Synthetic 75-12 peptide
PEPTDLYSYEQLSDS

SEQ ID NO: 3 - protein, 17 aa, Artificial Sequence
  Description of Artificial Sequence Synthetic 75-12 peptide
LEPEATDLYSYEQLSDS

SEQ ID NO: 4 - protein, 13 aa, Artificial Sequence
  Description of Artificial Sequence Synthetic 75-12 peptide
PETTDLHSYEQLG

SEQ ID NO: 5 - protein, 17 aa, Artificial Sequence
  Description of Artificial Sequence Synthetic 75-12 peptide
LHPEPTDLFSYEQLSDS

SEQ ID NO: 6 - protein, 15 aa, Artificial Sequence
  Description of Artificial Sequence Synthetic consensus peptide
  MOD_RES (3)..(3): Thr, Pro or Ala
  MOD_RES (7)..(7): Tyr, His or Phe
  MOD_RES (13)..(13): Asn, Ser or Gly
PEXTDLXSYEQLXDS

SEQ ID NO: 7 - protein, 15 aa, Artificial Sequence
  Description of Artificial Sequence Synthetic 75-12 peptide
NYEEVDLVSYEQLPD

SEQ ID NO: 8 - protein, 14 aa, Artificial Sequence
  Description of Artificial Sequence Synthetic 42-3 peptide
GTLGIVSPISSQKP

SEQ ID NO: 9 - protein, 17 aa, Artificial Sequence
  Description of Artificial Sequence Synthetic 143-7 peptide
MHGKVPTLQDVVLELTP

SEQ ID NO: 10 - protein, 17 aa, Artificial Sequence
  Description of Artificial Sequence Synthetic 143-7 peptide
MHGPKATLQDIVLHLEP

SEQ ID NO: 11 - protein, 15 aa, Artificial Sequence
  Description of Artificial Sequence Synthetic 143-7 peptide
GPKATLQDIVLHLEP

SEQ ID NO: 12 - protein, 15 aa, Artificial Sequence
  Description of Artificial Sequence Synthetic 143-7 peptide
MHGPKATLSDIVLDL

SEQ ID NO: 13 - protein, 13 aa, Artificial Sequence
  Description of Artificial Sequence Synthetic 143-7 peptide
NPTLREYILDLHP

SEQ ID NO: 14 - protein, 17 aa, Artificial Sequence
  Description of Artificial Sequence Synthetic consensus peptide
  MOD_RES (4)..(4): Lys, Pro or absent
  MOD_RES (5)..(5): Asn, Lys or Val
  MOD_RES (6)..(6): Pro or Ala
  MOD_RES (9)..(9): Gln, Ser or Arg
  MOD_RES (10)..(10): Asp or Glu
  MOD_RES (11)..(11): Val, Ile or Tyr
  MOD_RES (12)..(12): Val or Ile
  MOD_RES (14)..(14): Glu, His or Asp
  MOD_RES (16)..(16): Thr, Glu, His or absent
MHGXXXTLXXXXLXLXP

SEQ ID NO: 15 - protein, 19 aa, Artificial Sequence
  Description of Artificial Sequence Synthetic 21-10 peptide
LDLQPETTDLYSYEQLNDS

SEQ ID NO: 16 - protein, 21 aa, Artificial Sequence
  Description of Artificial Sequence Synthetic 21-10 peptide
YVLDLQPKATDLHSYEQLPDS

SEQ ID NO: 17 - protein, 17 aa, Artificial Sequence
  Description of Artificial Sequence Synthetic 21-10 peptide
LDLYPEPTDLYSYEQLS

SEQ ID NO: 18 - protein, 15 aa, Artificial Sequence
  Description of Artificial Sequence Synthetic 21-10 peptide
LDLEPEATDLYSYEQ

SEQ ID NO: 19 - protein, 21 aa, Artificial Sequence
  Description of Artificial Sequence Synthetic 21-10 peptide
YILDLQPETTDLHSYEQLGDS

SEQ ID NO: 20 - protein, 17 aa, Artificial Sequence
  Description of Artificial Sequence Synthetic 21-10 peptide
LDLHPEPTDLFSYEQLS

SEQ ID NO: 21 - protein, 17 aa, Artificial Sequence
  Description of Artificial Sequence Synthetic consensus peptide
  MOD_RES (4)..(4): Gln, Tyr, Glu or His
  MOD_RES (6)..(6): Glu or Lys
  MOD_RES (7)..(7): Thr, Ala or Pro
  MOD_RES (11)..(11): Tyr, His or Phe
  MOD_RES (17)..(17): Asn, Pro, Ser, Gly or absent
LDLXPXXTDLXSYEQLX

SEQ ID NO: 22 - protein, 19 aa, Artificial Sequence
  Description of Artificial Sequence Synthetic 57-4 peptide
LDLQPETTDLYSYEQLNDS

SEQ ID NO: 23 - protein, 17 aa, Artificial Sequence
  Description of Artificial Sequence Synthetic 57-4 peptide
LQPKATDLHSYEQLPDS

SEQ ID NO: 24 - protein, 17 aa, Artificial Sequence
  Description of Artificial Sequence Synthetic 57-4 peptide
LDLYPEPTDLYSYEQLS

SEQ ID NO: 25 - protein, 19 aa, Artificial Sequence
  Description of Artificial Sequence Synthetic 57-4 peptide
LDLEPEATDLYSYEQLSDS

SEQ ID NO: 26 - protein, 17 aa, Artificial Sequence
  Description of Artificial Sequence Synthetic 57-4 peptide
LQPETTDLHSYEQLGDS

SEQ ID NO: 27 - protein, 19 aa, Artificial Sequence
  Description of Artificial Sequence Synthetic 57-4 peptide
LDLHPEPTDLFSYEQLSDS

SEQ ID NO: 28 - protein, 19 aa, Artificial Sequence
  Description of Artificial Sequence Synthetic consensus peptide
  MOD_RES (4)..(4): Gln, Tyr, Glu or His
  MOD_RES (6)..(6): Glu or Lys
  MOD_RES (7)..(7): Thr, Ala or Pro
  MOD_RES (11)..(11): Tyr, His or Phe
  MOD_RES (17)..(17): Asn, Pro, Ser or Gly
LDLXPXXTDLXSYEQLXDS

SEQ ID NO: 29 - protein, 15 aa, Artificial Sequence
  Description of Artificial Sequence Synthetic 57-4 peptide
EQLDSSEDEDEDEVD

SEQ ID NO: 30 - protein, 19 aa, Artificial Sequence
  Description of Artificial Sequence Synthetic 58-3 peptide
LDLQPETTDLYSYEQLNDS

SEQ ID NO: 31 - protein, 17 aa, Artificial Sequence
  Description of Artificial Sequence Synthetic 58-3 peptide
LQPKATDLHSYEQLPDS

SEQ ID NO: 32 - protein, 21 aa, Artificial Sequence
  Description of Artificial Sequence Synthetic 58-3 peptide
YVLDLYPEPTDLYSYEQLSDS

SEQ ID NO: 33 - protein, 17 aa, Artificial Sequence
  Description of Artificial Sequence Synthetic 58-3 peptide
LEPEATDLYSYEQLSDS

SEQ ID NO: 34 - protein, 15 aa, Artificial Sequence
  Description of Artificial Sequence Synthetic 58-3 peptide
LQPETTDLHSYEQLG

SEQ ID NO: 35 - protein, 19 aa, Artificial Sequence
  Description of Artificial Sequence Synthetic 58-3 peptide
LDLHPEPTDLFSYEQLSDS

SEQ ID NO: 36 - protein, 19 aa, Artificial Sequence
  Description of Artificial Sequence Synthetic consensus peptide
  MOD_RES (4)..(4): Gln, Tyr, Glu or His
  MOD_RES (6)..(6): Glu or Lys
  MOD_RES (7)..(7): Thr, Ala or Pro
  MOD_RES (11)..(11): Tyr, His or Phe
  MOD_RES (17)..(17): Asn, Pro, Ser or Gly
LDLXPXXTDLXSYEQLXDS

SEQ ID NO: 37 - protein, 15 aa, Artificial Sequence
  Description of Artificial Sequence Synthetic 58-3 peptide
EQLDSSEDEDEDEVD

SEQ ID NO: 38 - protein, 13 aa, Artificial Sequence
  Description of Artificial Sequence Synthetic 78-11 peptide
MRGNNPTLREYIL

SEQ ID NO: 39 - protein, 15 aa, Artificial Sequence
  Description of Artificial Sequence Synthetic 80-2 peptide
EEEDEIDGPAGQAEP

SEQ ID NO: 40 - protein, 13 aa, Artificial Sequence
  Description of Artificial Sequence Synthetic 80-2 peptide
VIDSPAGQAKPDT

SEQ ID NO: 41 - protein, 11 aa, Artificial Sequence
  Description of Artificial Sequence Synthetic consensus peptide
  MOD_RES (1)..(1): Glu or Val
  MOD_RES (4)..(4): Gly or Ser
  MOD_RES (10)..(10): Glu or Lys
XIDXPAGQAXP

SEQ ID NO: 42 - protein, 13 aa, Artificial Sequence
  Description of Artificial Sequence Synthetic 80-2 peptide
MDTLSFVSPLSAA

SEQ ID NO: 43 - protein, 15 aa, Artificial Sequence
  Description of Artificial Sequence Synthetic 84-2 peptide
EDVIDSPAGQAKPDT

SEQ ID NO: 44 - protein, 19 aa, Artificial Sequence
  Description of Artificial Sequence Synthetic 84-2 peptide
SSDEDEGLDRPDGQAQPAT

SEQ ID NO: 45 - protein, 19 aa, Artificial Sequence
  Description of Artificial Sequence Synthetic 84-2 peptide
DEEDTDGVDRPDGQAEQAT

SEQ ID NO: 46 - protein, 21 aa, Artificial Sequence
  Description of Artificial Sequence Synthetic 84-2 peptide
SSDEDEIGLDRPDGQAQPATA

SEQ ID NO: 47 - protein, 20 aa, Artificial Sequence
  Description of Artificial Sequence Synthetic consensus peptide
  MOD_RES (2)..(2): Ser, Asp or absent
  MOD_RES (3)..(3): Asp, Glu or absent
  MOD_RES (6)..(6): Glu, Thr or absent
  MOD_RES (7)..(7): Asp, Glu or Ile
  MOD_RES (8)..(8): Val or Gly
  MOD_RES (9)..(9): Ile, Leu or Val
  MOD_RES (13)..(13): Ala or Asp
  MOD_RES (17)..(17): Lys, Gln or Glu
  MOD_RES (18)..(18): Pro or Gln
SXXEDXXXXDRPXGQAXXAT

SEQ ID NO: 48 - protein, 13 aa, Artificial Sequence
  Description of Artificial Sequence Synthetic 19-1 peptide
VPTLQDVVLELTP

SEQ ID NO: 49 - protein, 13 aa, Artificial Sequence
  Description of Artificial Sequence Synthetic 19-1 peptide
MHGPKATLSDIVL

SEQ ID NO: 50 - protein, 9 aa, Artificial Sequence
  Description of Artificial Sequence Synthetic consensus peptide
  MOD_RES (1)..(1): Val or Lys
  MOD_RES (2)..(2): Pro or Ala
  MOD_RES (5)..(5): Gln or Ser
  MOD_RES (7)..(7): Val or Ile
XXTLXDXVL

SEQ ID NO: 51 - protein, 15 aa, Artificial Sequence
  Description of Artificial Sequence Synthetic 37-1 peptide
EQLEDSSEDEVDKVK

SEQ ID NO: 52 - protein, 21 aa, Artificial Sequence
  Description of Artificial Sequence Synthetic 37-1 peptide
QLNDSSEEEDEIDGPAGQAEP

SEQ ID NO: 53 - protein, 23 aa, Artificial Sequence
  Description of Artificial Sequence Synthetic 37-1 peptide
EQLSDSEEENDEIDGVNHQHLPA

SEQ ID NO: 54 - protein, 17 aa, Artificial Sequence
  Description of Artificial Sequence Synthetic 37-1 peptide
SSDEEDVIDSPAGQAKP

SEQ ID NO: 55 - protein, 24 aa, Artificial Sequence
  Description of Artificial Sequence Synthetic consensus peptide
  MOD_RES (4)..(4): Glu, Asn, Ser or absent
  MOD_RES (8)..(8): Glu or Asp
  MOD_RES (11)..(11): Asn or absent
  MOD_RES (17)..(17): Lys, Gly or Ser
  MOD_RES (18)..(18): Val or Pro
  MOD_RES (19)..(19): Lys, Ala or Asn
  MOD_RES (20)..(20): Gly, His or absent
  MOD_RES (22)..(22): Ala, His or absent
  MOD_RES (23)..(23): Glu, Leu, Lys or absent
EQLXDSSXEEXDEVIDXXXXQXXP

SEQ ID NO: 56 - protein, 19 aa, Artificial Sequence
  Description of Artificial Sequence Synthetic 37-1 peptide
SSEDEDEDEVDHLQERPQQ

SEQ ID NO: 57 - protein, 23 aa, Artificial Sequence
  Description of Artificial Sequence Synthetic 37-1 peptide
VSHEQLGDSDDEIDEPDHAVNHH

SEQ ID NO: 58 - protein, 13 aa, Artificial Sequence
  Description of Artificial Sequence Synthetic 55-11 peptide
LEDSSEDEVDKVD

SEQ ID NO: 59 - protein, 17 aa, Artificial Sequence
  Description of Artificial Sequence Synthetic 55-11 peptide
DSEEENDEIDGVNHQHL

SEQ ID NO: 60 - protein, 15 aa, Artificial Sequence
  Description of Artificial Sequence Synthetic consensus peptide
  MOD_RES (3)..(3): Ser or absent
  MOD_RES (5)..(5): Glu or absent
  MOD_RES (6)..(6): Glu or absent
  MOD_RES (7)..(7): Asn or absent
  MOD_RES (10)..(10): Val or absent
  MOD_RES (11)..(11): Ile or absent
  MOD_RES (13)..(13): Lys or Gly
  MOD_RES (15)..(15): Asp or Asn
DSXEXXXDEXXDXVX

SEQ ID NO: 61 - protein, 21 aa, Artificial Sequence
  Description of Artificial Sequence Synthetic 55-11 peptide
LHLEPQNELDPVDLLSYEQLS

SEQ ID NO: 62 - protein, 19 aa, Artificial Sequence
  Description of Artificial Sequence Synthetic 55-11 peptide
LEPQNYEEVDLVSYEQLPD

SEQ ID NO: 63 - protein, 21 aa, Artificial Sequence
  Description of Artificial Sequence Synthetic 55-11 peptide
LELSPSNEIEPVDLVSHEQLG

SEQ ID NO: 64 - protein, 13 aa, Artificial Sequence
  Description of Artificial Sequence Synthetic 55-11 peptide
LYPYNEIQPVDLV

SEQ ID NO: 65 - protein, 22 aa, Artificial Sequence
  Description of Artificial Sequence Synthetic consensus peptide
  MOD_RES (2)..(2): His, Glu or absent
  MOD_RES (4)..(4): Glu, Ser or Tyr
  MOD_RES (6)..(6): Gln, Ser or Tyr
  MOD_RES (9)..(9): Leu, Tyr or Ile
  MOD_RES (10)..(10): Asp, Glu or Gln
  MOD_RES (11)..(11): Pro or Glu
  MOD_RES (15)..(15): Leu or Val
  MOD_RES (17)..(17): Tyr, His or absent
  MOD_RES (21)..(21): Ser, Pro, Gly or absent
  MOD_RES (22)..(22): Asp or absent
LXLXPXNEXXXVDLXSXEQLXX

SEQ ID NO: 66 - protein, 13 aa, Artificial Sequence
  Description of Artificial Sequence Synthetic 38-5 peptide
FSSQSESTLRLSV

SEQ ID NO: 67 - protein, 13 aa, Artificial Sequence
  Description of Artificial Sequence Synthetic 146-8 peptide
MHGRLVTLKDIVL

SEQ ID NO: 68 - protein, 17 aa, Artificial Sequence
  Description of Artificial Sequence Synthetic 146-8 peptide
MHGKVPTLQDVVLELTP

SEQ ID NO: 69 - protein, 13 aa, Artificial Sequence
  Description of Artificial Sequence Synthetic consensus peptide
  MOD_RES (4)..(4): Arg or Lys
  MOD_RES (5)..(5): Leu or Val
  MOD_RES (6)..(6): Val or Pro
  MOD_RES (9)..(9): Lys or Gln
  MOD_RES (11)..(11): Ile or Val
MHGXXXTLXDXVL

SEQ ID NO: 70 - protein, 13 aa, Artificial Sequence
  Description of Artificial Sequence Synthetic 146-8 peptide
PLTQHYQILTSSS

SEQ ID NO: 71 - protein, 13 aa, Artificial Sequence
  Description of Artificial Sequence Synthetic 146-8 peptide
RAHYNIVTFSSKS

SEQ ID NO: 72 - protein, 13 aa, Artificial Sequence
  Description of Artificial Sequence Synthetic 146-8 peptide
TSNYNIVTFSSQS

SEQ ID NO: 73 - protein, 13 aa, Artificial Sequence
  Description of Artificial Sequence Synthetic 146-8 peptide
ATANYYIVTSSYT

SEQ ID NO: 74 - protein, 13 aa, Artificial Sequence
  Description of Artificial Sequence Synthetic consensus peptide
  MOD_RES (1)..(1): Thr or Arg
  MOD_RES (2)..(2): Gln, Ala or Ser
  MOD_RES (3)..(3): His or Asn
  MOD_RES (5)..(5): Gln, Asn or Tyr
  MOD_RES (7)..(7): Leu or Val
  MOD_RES (12)..(12): Ser, Lys, Gln or Tyr
  MOD_RES (13)..(13): Ser, Thr or absent
XXXYXIXTFSSXX

SEQ ID NO: 75 - protein, 15 aa, Artificial Sequence
  Description of Artificial Sequence Synthetic 146-8 peptide
LNTLSFVSPWSASQQ

SEQ ID NO: 76 - protein, 13 aa, Artificial Sequence
  Description of Artificial Sequence Synthetic 146-8 peptide
TLSFVSPWSATNQ

SEQ ID NO: 77 - protein, 13 aa, Artificial Sequence
  Description of Artificial Sequence Synthetic 146-8 peptide
ALTVTSPLSASSN

SEQ ID NO: 78 - protein, 13 aa, Artificial Sequence
  Description of Artificial Sequence Synthetic consensus peptide
  MOD_RES (1)..(1): Thr or Ala
  MOD_RES (3)..(3): Ser or Thr
  MOD_RES (4)..(4): Phe or Val
  MOD_RES (5)..(5): Val or Thr
  MOD_RES (8)..(8): Trp or Leu
  MOD_RES (11)..(11): Ser or Thr
  MOD_RES (12)..(12): Gln, Asn or Ser
  MOD_RES (13)..(13): Gln or Asn
XLXXXSPXSAXXX

SEQ ID NO: 79 - protein, 13 aa, Artificial Sequence
  Description of Artificial Sequence Synthetic 146-8 peptide
DRPDGQAEQATSN

SEQ ID NO: 80 - protein, 19 aa, Artificial Sequence
  Description of Artificial Sequence Synthetic 159-1 peptide
MHGRLVTLKDIVLDLQPPD

SEQ ID NO: 81 - protein, 18 aa, Artificial Sequence
  Description of Artificial Sequence Synthetic 159-1 peptide
GPKATLQDIVLHLLEPQN

SEQ ID NO: 82 - protein, 21 aa, Artificial Sequence
  Description of Artificial Sequence Synthetic 159-1 peptide
MHGKVPTLQDVVLELTPQTEI

SEQ ID NO: 83 - protein, 15 aa, Artificial Sequence
  Description of Artificial Sequence Synthetic 159-1 peptide
MHGPKATLSDIVLDL

SEQ ID NO: 84 - protein, 20 aa, Artificial Sequence
  Description of Artificial Sequence Synthetic consensus peptide
  MOD_RES (4)..(4): Arg, Pro or Lys
  MOD_RES (5)..(5): Leu, Lys or Val
  MOD_RES (6)..(6): Val, Ala or Pro
  MOD_RES (9)..(9): Lys, Gln or Ser
  MOD_RES (11)..(11): Ile or Val
  MOD_RES (14)..(14): Asp, His or Glu
  MOD_RES (16)..(16): Gln, Leu, Thr or absent
  MOD_RES (17)..(17): Pro, Glu or absent
  MOD_RES (18)..(18): Pro, Gln or absent
  MOD_RES (19)..(19): Asp, Gln, Thr or absent
  MOD_RES (20)..(20): Asn, Glu or absent
MHGXXXTLXDXVLXLXXXXX

SEQ ID NO: 85 - protein, 13 aa, Artificial Sequence
  Description of Artificial Sequence Synthetic 159-1 peptide
YSYEQLNDSSEEE

SEQ ID NO: 86 - protein, 15 aa, Artificial Sequence
  Description of Artificial Sequence Synthetic 159-1 peptide
LQPKATDLHSYEQLP

SEQ ID NO: 87 - protein, 27 aa, Artificial Sequence
  Description of Artificial Sequence Synthetic 159-1 peptide
LDLYPEPTDLYSYEQLSDSSDEDEGLD

SEQ ID NO: 88 - protein, 17 aa, Artificial Sequence
  Description of Artificial Sequence Synthetic 159-1 peptide
LEPEATDLYSYEQLSDS

SEQ ID NO: 89 - protein, 21 aa, Artificial Sequence
  Description of Artificial Sequence Synthetic consensus peptide
  MOD_RES (2)..(2): Gln, Tyr, Glu or absent
  MOD_RES (4)..(4): Lys, Glu or absent
  MOD_RES (5)..(5): Ala, Pro or absent
  MOD_RES (9)..(9): Tyr or His
  MOD_RES (15)..(15): Asn, Pro or Ser
  MOD_RES (19)..(19): Glu, Asp or absent
  MOD_RES (21)..(21): Glu, Asp or absent
LXPXXTDLXSYEQLXDSSXEX

SEQ ID NO: 90 - protein, 19 aa, Artificial Sequence
  Description of Artificial Sequence Synthetic 128-3 peptide
MHGRLVTLKDIVLDLQPPD

SEQ ID NO: 91 - protein, 19 aa, Artificial Sequence
  Description of Artificial Sequence Synthetic 128-3 peptide
MHGPKATLQDIVLHLEPQN

SEQ ID NO: 92 - protein, 19 aa, Artificial Sequence
  Description of Artificial Sequence Synthetic 128-3 peptide
MHGPQATLQEIVLHLEPQN

SEQ ID NO: 93 - protein, 23 aa, Artificial Sequence
  Description of Artificial Sequence Synthetic 128-3 peptide
MHGKVPTLQDVVLELTPQTEIDL

SEQ ID NO: 94 - protein, 15 aa, Artificial Sequence
  Description of Artificial Sequence Synthetic 128-3 peptide
MHGPKATLSDIVLDL

SEQ ID NO: 95 - protein, 13 aa, Artificial Sequence
  Description of Artificial Sequence Synthetic 128-3 peptide
TLQEIVLDLYPYN

SEQ ID NO: 96 - protein, 19 aa, Artificial Sequence
  Description of Artificial Sequence Synthetic consensus peptide
  MOD_RES (4)..(4): Arg, Pro, Lys or absent
  MOD_RES (5)..(5): Leu, Lys, Gln, Val or absent
  MOD_RES (6)..(6): Val, Ala, Pro or absent
  MOD_RES (9)..(9): Lys, Gln or Ser
  MOD_RES (10)..(10): Asp or Glu
  MOD_RES (11)..(11): Ile or Val
  MOD_RES (14)..(14): Asp, His or Glu
  MOD_RES (16)..(16): Gln, Glu, Thr, Tyr or absent
  MOD_RES (18)..(18): Pro, Gln, Tyr or absent
  MOD_RES (19)..(19): Asp, Asn, Thr or absent
MHGXXXTLXXXVLXLXPXX

SEQ ID NO: 97 - protein, 19 aa, Artificial Sequence
  Description of Artificial Sequence Synthetic 167-5 peptide
MHGRLVTLKDIVLDLQPPD

SEQ ID NO: 98 - protein, 17 aa, Artificial Sequence
  Description of Artificial Sequence Synthetic 167-5 peptide
GPKATLQDIVLHLEPQN

SEQ ID NO: 99 - protein, 23 aa, Artificial Sequence
  Description of Artificial Sequence Synthetic 167-5 peptide
MHGKVPTLQDVVLELTPQTEIDL

SEQ ID NO: 100 - protein, 15 aa, Artificial Sequence
  Description of Artificial Sequence Synthetic 167-5 peptide
MHGPKATLSDIVLDL

SEQ ID NO: 101 - protein, 19 aa, Artificial Sequence
  Description of Artificial Sequence Synthetic consensus peptide
  MOD_RES (4)..(4): Arg, Pro or Lys
  MOD_RES (5)..(5): Leu, Lys or Val
  MOD_RES (6)..(6): Val, Ala or Pro
  MOD_RES (9)..(9): Lys, Gln or Ser
  MOD_RES (11)..(11): Ile or Val
  MOD_RES (14)..(14): His, Glu or Asp
  MOD_RES (16)..(16): Gln, Glu, Thr or absent
  MOD_RES (18)..(18): Pro, Gln or absent
  MOD_RES (19)..(19): Asp, Asn, Thr or absent
MHGXXXTLXDXVLXLXPXX

(19) United States
(12) Patent Application Publication
(10) Pub. No.: US 2018/0259523 A1
(43) Pub. Date: Sep. 13, 2018

(54) IMMUNOLOGICAL TEST FOR THE DETECTION OF E7 ONCOPROTEINS IN BIOLOGICAL SAMPLES

(71) Applicants: Mikrogen GmbH, Neuried (DE); Österreichische Akademie der Wissenschaften, Vienna (AT)

(72) Inventors: Pidder JANSEN-DÜRR, Innsbruck (AT); Oliver BÖCHER, Neuried (DE); Isabel KOCH, Munich (DE); Erwin SOUTSCHEK, Berg (DE)

(73) Assignees: Mikrogen GmbH, Neuried (DE); Österreichische Akademie der Wissenschaften, Vienna (AT)

(21) Appl. No.: 15/517,150

(22) PCT Filed: Oct. 30, 2015
(86) PCT No.: PCT/EP2015/075214

§ 371 Date: Apr. 5, 2017

Related U.S. Application Data
(60) Provisional application No. 62/073,116, filed on Oct. 31, 2014.

(30) Foreign Application Priority Data
Oct. 31, 2014 (EP) 14191193.3

(51) Int. Cl. G01N 33/569; G01N 33/574; C07K 16/08; G01N 33/541; G01N 33/531

(52) U.S. Cl. CPC G01N 33/56983; G01N 33/57411; C07K 16/084; G01N 33/541; G01N 2800/56; G01N 2333/025; G01N 2800/7028; G01N 2800/50; G01N 2800/52; G01N 33/531

(57) ABSTRACT

The present invention relates to a diagnostic test for the detection of an E7 protein of a human papilloma virus in a biological sample wherein a sandwich ELISA as capture antibody at least two different rabbit monoclonal antibodies which bind to at least two different epitopes are used and as detection antibody at least two different polyclonal anti E7 antibodies are used.

### PRIORITY

This application corresponds to the U.S. national phase of International Application No. PCT/EP2015/075214, filed Oct. 30, 2015, which, in turn, claims priority to European Patent Application No. 14.191193.3 filed Oct. 31, 2014 and U.S. Provisional Application No. 62/073,116 filed Oct. 31, 2014, the contents of which are incorporated by reference herein in their entirety.

### FIELD OF THE INVENTION

The present invention relates to diagnostic test kits and methods for the detection of an E7 protein of a human papilloma virus in a biological sample.

### BACKGROUND OF THE INVENTION

Cervical cancer is one of the leading causes of cancer morbidity and mortality in women with more than 98% related to a human papilloma virus (HPV) infection origin. Infection with specific subtypes of HPV has been strongly implicated in cervical carcinoma genesis. Human papilloma viruses have circular, double-stranded DNA genomes that are approximately 8 kb in size and encode eight genes of which E6 and E7 have transforming properties. Viral E6 and E7 oncoproteins are necessary for malignant conversion. E7 plays a central role in both the viral life cycle and carcinogenic transformation (McLaughlin-Drubin et al., Virology 384 (2009), pp. 335-344). There are several different strains of HPV whereby some strains such as in particular HPV-16 and HPV-18 are known as high-risk type HPVs. On the other hand there are also HPV strains, such as HPV-6 and HPV-11 which are designated as low-risk type HPVs. Furthermore, there are several other HPV strains such as HPV-31, 33, 35, 39, 45, 51, 52, 56, 58 and 59 which bear a rather high risk for the patient. Those strains occur, however, with a lower frequency. It can be assumed that about 80% of cervical cancer worldwide are associated with only four types (16, 18, 31 and 45). In other 15% of cancer HPV types 33, 35 and 52 are detected.

US 2005/0142541 discloses a detection reagent for E6 proteins of high-risk HPVs comprising a mixture of monoclonal antibodies which specifically bind to E6 proteins of at least three different oncogenic HPV strains. US 2013/0029322 and US 2007/0166699, respectively, disclose assays for E7 proteins of the high-risk HPV types. Although the teaching of this US patent application allows the detection of several high-risk strains it is, however, not possible to detect all HPV strains in one assay.

Since in different patients different HPV strains may be the cause for cervical cancer it is one object of the present invention to provide a diagnostic method whereby in one test at least 95%, preferably 99% or more of all high-risk types of HPV can be detected. The test should, however, not detect low-risk strains HPV-6 and HPV-11, respectively.

### SUMMARY OF THE INVENTION

The present invention relates to a diagnostic test for the detection of an E7 protein of a human papilloma virus in a biological sample whereby in a sandwich ELISA as capture antibody at least two different rabbit monoclonal antibodies are used which bind to at least two different epitopes. As detection antibody at least two different polyclonal anti E7 antibodies are used.

The immunological test of the present invention is based on the principle of a so-called sandwich ELISA. In the test “sandwich” the antigen can be considered as the “ham” and the capture and detection antibodies are the two sides of the roll. In a sandwich ELISA there are capture antibodies, which are usually attached to the surface of the reaction well. In the present invention the capture antibodies are monoclonal antibodies which were raised against different E7 proteins obtained preferably recombinantly from high-risk HPV strains.

### BRIEF DESCRIPTION OF THE FIGURES

FIG. 1 depicts the test principle of a preferred embodiment of the diagnostic test method of the present invention.

FIGS. 2a and 2b depict the results of the experiments of Example 1. FIG. 2a depicts the results for combinations 1, 2, and 3 and FIG. 2b depicts the results for combinations 4, 5, and 6

FIG. 3 depicts the detection results for 12 high-risk HPV types assayed in the one-well format described in Example 1.

FIG. 4 depicts the titration results for the E7 proteins detected in the one-well format described in Example 1.

FIG. 5 depicts the results of various one-well control experiments.

FIGS. 6a and 6b depict the results of the experiments of Example 2. FIG. 6a depicts the results for the various one-well systems: wells 1, 2, and 3 independently. FIG. 6b depicts the results for the three-well system: wells 1, 2, and 3 together.

FIG. 7 depicts the results of various control experiments with well 1.

FIG. 8 presents results confirming the utility of the diagnostic test method of the present invention in detecting clinically abnormal smears. In particular, the data in FIG. 8 demonstrate that the E7 signal in HPV DNA negative samples without clinical findings was in the range of the background signal, whereas the HVP DNA positive, clinically abnormal samples, clearly displayed E7 content above background.

### DETAILED DESCRIPTION OF THE PREFERRED EMBODIMENTS

There are several different techniques known in the art how monoclonal antibodies can be produced. Usually animals are immunized and antibody producing cells of the immunized animal are fused with tumor cells. Subsequently the antibody producing hybridoma cells are singled out in order to obtain a hybridoma cell line which produces only one type of monoclonal antibodies. In general most monoclonal antibodies are produced using the mouse system. It is, however, an important aspect of the present invention that the monoclonal antibodies are produced from rabbits.

In the diagnostic test the biological sample to be tested is brought into the wells which are already coated with the monoclonal capture antibodies. The biological sample is preferably a sample obtained from the cervix, preferably the epithelial cells of the cervix. Since human papilloma virus can also be involved in other cancer forms such as head and neck cancer (oropharyngial cancer) or anal cancer the biological sample can also be obtained from patients suffering from such cancers. For therapy it is important to know whether HPV and in particular high-risk strains thereof are involved in such cancer. From the diagnostic view it is desirable to detect all high-risk strains in one convenient assay.

The antigen, namely the HPV E7 protein (if present), binds to the capture antibodies. Afterwards unbound material is washed away. For the detection of the E7 protein a so-called detection antibody is used. According to the present invention the “detection antibodies” are polyclonal antibodies obtained by immunization of an animal with specific E7 proteins. According to preferred embodiments of the invention different polyclonal antibodies are used which are obtained by immunization with different antigens.

In one preferred embodiment of the present invention the animal is immunized with recombinantly produced HPV-16 E7 in order to produce one type of polyclonal antibody. Another polyclonal antibody is obtained by immunizing the animal with recombinantly produced HPV-18 E7. In a further preferred embodiment the polyclonal antiserum is obtained by immunizing an animal with a mixture of the E7 proteins derived from different HPV types, preferably types 39, 51, 56 and 59. Alternatively also mixtures comprising E7 proteins of HPV types 33, 35 and 52 can be used. In an especially preferred embodiment a mixture of three or four different strains is used in a ratio of about 1:1:1:1 of E7 proteins of different HPV types. A mixture of 39, 51, 56 and 59 is especially preferred. The mixture contains between 20 and 30% of each of the four proteins when four proteins are used whereby necessarily 100% are obtained. Slight variations of the relationships are acceptable.

In a particularly preferred embodiment the three (or more) different polyclonal antibodies as described above are used together for the analysis of each sample.

The detection antibody is responsible for the test signal. In a preferred embodiment the polyclonal antibodies obtained from the animal are purified and biotinylated. The biotinylation allows the link of the detection antibody to a label which forms a detectable signal.

In preferred embodiments the label forms the signal which is preferably created by the action of an enzyme which converts a precursor to a product which results for example in a colour change of the reaction medium. Very frequently ELISA tests are performed on titer plates having several (e.g. 96) wells. The titer plates are part of the kit for performing the immunological test.

According to the present invention it is possible either to attach as capture antibody several different rabbit monoclonal antibodies into one single well or alternatively the rabbit monoclonal antibodies can be attached to different wells, whereby, however, for the detection of the complete results several wells coated with different monoclonal antibodies have to be evaluated together for a single biological sample.

The advantage of putting several, preferably three to five rabbit monoclonal antibodies into one well and to use also three different polyclonal detection antibodies into one well is the simplicity of the method. It is also possible to use more than five different rabbit monoclonal antibodies in one well, but when the number of different mAbs is too high the percentage of each mAb will be too low for a reliable detection.

In an alternative embodiment it is possible to foresee for one biological sample three different test wells which are coated with different combinations of rabbit monoclonal antibodies. These three test wells foreseen for one biological sample can be reacted with the same amount of antigen. For the detection of the antigen different polyclonal antibodies can be used in the three test wells. The advantage of separating the biological sample into three different test wells and performing the test with different polyclonal antibodies is that the diagnostic result may be more precise insofar as it can be better determined which specific type of HPV is detected in the biological sample.

The downside of separating the sample into three different wells is that the results have to be combined thereafter which cause an additional step. The advantage of using several different capture antibodies together with several different polyclonal antibodies as detection means in one well is that a result can be obtained easily insofar as it can be detected whether in the biological sample there is a high-risk variant of HPV or not.

According to the present invention the monoclonal antibodies are preferably obtained from rabbit. The polyclonal antibodies could also be obtained from other animals which are usually used for the production of polyclonal antibodies, namely rabbits, sheep, horses or goats whereby goats are particularly preferred.

The term “polyclonal antibodies”, which are used as detection antibodies in the diagnostic test of the present invention, designates a purified fraction of antibodies obtained from the blood of an immunized animal. Usually the antigen is applied intravenously, intradermally, intramuscularly, or subcutaneously to the animal, preferably together with an adjuvant which triggers the formation of antibodies.

Frequently the application of the antigen occurs three to four times whereby the time difference between each application (booster) of the antigen is 2-6 weeks. When the antibody titer has reached the desired level a large amount of blood is taken from the animal. The serum is obtained from the blood and subsequently the antibodies are separated from the serum. This can be done with suitable separation means which allow the enrichment of the antibodies (e.g. suitable columns).

The antigen which is used for the immunization of the animals and which is also used for the production of the rabbit monoclonal antibodies is usually recombinant material. Since the sequences of the different E7 proteins from different strains are known the genes coding for those sequences can be cloned in suitable expression vectors and the proteins can be expressed in suitable hosts (e.g. E. coli). After expression in the host the recombinantly produced E7 proteins are purified nearly to homogeneity. It is desired to avoid any impurities since such impurities may elicit unspecific antibodies.

One embodiment of the present invention relates to diagnostic test kits which are suitable for performing the diagnostic test of the present invention. Usually such kits contain the rabbit monoclonal antibodies which function as capture antibodies linked to a solid phase preferably in a reaction well. Alternatively, however, the capture antibodies can be linked to beads which may be made from plastic material (e.g. sterol). The detection antibody is usually contained within the test kit in a suitable form. In a preferred embodiment the detection antibody is present in a ready to use form or in a lyophilized form which can be reconstituted with suitable buffer solution.

In order to characterize the rabbit monoclonal antibodies further an epitope mapping has been performed. The monoclonal antibodies as further described in the Table 1 and their epitope sequences to which they bind are preferably used in the diagnostic test methods of the present invention and the test kits which are designed for performing the invention. Table 1 discloses also preferred consensus sequences wherein a “*” stands for an amino acid which may vary whereby the “*” stands, however, preferably for an amino acid which is disclosed at the relevant epitope sequence from which such consensus sequence is derived. The following epitopes to which the monoclonal antibodies bind have been identified:

TABLE 1
Sequences to which the Rabbit Monoclonal Antibodies Bind

| | AA | | | |
|---|---|---|---|---|
| RabMab | position | HPV | Sequence | Seq.ID |
| 75-12 | | 16E7 | --PETTDLYSYEQLNDS | 1 |
| | | 33E7 | --PEPTDLYSYEQLSDS | 2 |
| | | 35E7 | LEPEATDLYSYEQLSDS | 3 |
| | | 52E7 | --PETTDLHSYEQLG-- | 4 |
| | | 58E7 | LHPEPTDLFSYEQLSDS | 5 |
| | 15-31 | Consensus | --PE*TDL*SYEQL*DS | 6 |
| | 16-30 | 59E7 | -NYEEVDLVSYEQLPD- | 7 |
| 42-3^{#} | 85-98 | 16E7 | GTLGIVSPISSQKP | 8 |
| 143-7 | | 56E7 | MHGKVPTLQDVVLELTP | 9 |
| | | 18E7 | MHGPKATLQDIVLHLEP | 10 |
| | | 45E7 | --GPKATLQDIVLHLEP | 11 |
| | | 59E7 | MHGPKATLSDIVLDL-- | 12 |
| | | 58E7 | ----NPTLREYILDLHP | 13 |
| | 1-17 | Consensus | MHG***TL****L*L*P | 14 |
| 21-10 | | 16E7 | --LDLQPETTDLYSYEQLNDS | 15 |
| | | 31E7 | YVLDLQPKATDLHSYEQLPDS | 16 |
| | | 33E7 | --LDLYPEPTDLYSYEQLS-- | 17 |
| | | 35E7 | --LDLEPEATDLYSYEQ---- | 18 |
| | | 52E7 | YILDLQPETTDLHSYEQLGDS | 19 |
| | | 58E7 | --LDLHPEPTDLFSYEQLS-- | 20 |
| | 13-28 | Consensus | --LDL*P**TDL*SYEQL*-- | 21 |
| 57-4 | | 16E7 | LDLQPETTDLYSYEQLNDS | 22 |
| | | 31E7 | --LQPKATDLHSYEQLPDS | 23 |
| | | 33E7 | LDLYPEPTDLYSYEQLS-- | 24 |
| | | 35E7 | LDLEPEATDLYSYEQLSDS | 25 |
| | | 52E7 | --LQPETTDLHSYEQLGDS | 26 |
| | | 58E7 | LDLHPEPTDLFSYEQLSDS | 27 |
| | 13-31 | Consensus | LDL*P**TDL*SYEQL*DS | 28 |
| | 26-40 | 56E7 | -------------EQL-DSSEDEDEDEVD | 29 |
| 58-3 | | 16E7 | --LDLQPETTDLYSYEQLNDS | 30 |
| | | 31E7 | ----LQPKATDLHSYEQLPDS | 31 |
| | | 33E7 | YVLDLYPEPTDLYSYEQLSDS | 32 |
| | | 35E7 | ----LEPEATDLYSYEQLSDS | 33 |
| | | 52E7 | ----LQPETTDLHSYEQLG-- | 34 |
| | | 58E7 | --LDLHPEPTDLFSYEQLSDS | 35 |
| | 11-31 | Consensus | --LDL*P**TDL*SYEQL*DS | 36 |
| | 26-40 | 56E7 | ---------------EQL-DSSEDEDEDEVD | 37 |
| 78-11 | 1-13 | 58E7 | MRGNNPTLREYIL | 38 |
| 80-2 | | 16E7 | EEEDEIDGPAGQAEP-- | 39 |
| | | 31E7 | ----VIDSPAGQAKPDT | 40 |
| | 31-51 | Consensus | ----*ID*PAGQA*P-- | 41 |
| | 93-105 | 59E7 | MDTLSFVSPLSAA | 42 |
| 84-2 | | 31E7 | -----EDVIDSPAGQAKPDT- | 43 |
| | | 33E7 | SSDED-EGLDRPDGQAQPAT- | 44 |
| | | 52E7 | -DEEDTDGVDRPDGQAEQAT- | 45 |
| | | 58E7 | SSDEDEIGLDRPDGQAQPATA | 46 |
| | 31-51 | Consensus | S**ED****DRP*GQA**AT- | 47 |
| 19-1 | | 56E7 | ----VPTLQDVVLELTP | 48 |
| | | 59E7 | MHGPKATLSDIVL---- | 49 |
| | 1-17 | Consensus | ----**TL*D*VL---- | 50 |
| 31-7 | | 11E7 | EQLEDSSE---DEV-DKVK------ | 51 |
| | | 16E7 | -QLNDSSEEE-DE-IDGPAGQAEP- | 52 |
| | | 18E7 | EQLSDS-EEENDE-IDGVNHQHLPA | 53 |
| | | 31E7 | -----SSDEE-D-VIDSPAGQAKP- | 54 |
| | 27-51 | Consensus | EQL*DSS*EE*DEVID****Q**P- | 55 |
| | 31-50 | 56E7 | ----SSEDEDEDEV-DHLQERPQQ- | 56 |
| | 27-51 | 68E7 | VSHEQLGDSD-DE-IDEPDHAVNHH | 57 |
| 55-11 | | 11E7 | LEDSSE---DEV-DKVD---- | 58 |
| 2 | | 18E7 | --DS-EEENDE-IDGVNHQHL | 59 |
| consensus | 13-45 | Consensus | --DS*E***DE**D*V*---- | 60 |
| regions | | 45E7 | LHLEPQNELDPVDLLSYEQLS- | 61 |
| | | 59E7 | --LEPQN-YEEVDLVSYEQLPD | 62 |
| | | 68E7 | LELSPSNEIEPVDLVSHEQLG- | 63 |
| | | 70E7 | --LYPYNEIQPVDLV------- | 64 |
| | 13-45 | Consensus | L*L*P*NE***VDL*S*EQL** | 65 |
| 38-5^{$} | 57-69 | 31E7 | FSSQSESTLRLSV | 66 |
| 146-8 | | 11E7 | MHGRLVTLKDIVL---- | 67 |
| 3 | | 56E7 | MHGKVPTLQDVVLELTP | 68 |
| consensus | 1-17 | Consensus | MHG***TL*D*VL---- | 69 |
| regions | | 11E7 | PLTQHYQILT-SSS- | 70 |
| | | 16E7 | --RAHYNIVTFSSKS | 71 |
| | | 31E7 | --TSNYNIVTFSSQS | 72 |
| | | 58E7 | -ATANYYIVT-SSYT | 73 |
| | 49-61 | Consensus | --***Y*I*TFSS** | 74 |
| | | 18E7 | LNTLSFVSPWSASQQ | 75 |
| | | 45E7 | --TLSFVSPWSATNQ | 76 |
| | | 56E7 | --ALTVTSPLSASSN | 77 |
| | 91-105 | Consensus | --*L***SP*SA*** | 78 |
| | 41-53 | 52E7 | DRPDGQAEQATSN | 79 |
| 159-1 | | 11E7 | MHGRLVTLKDIVLDLQPPD-- | 80 |
| 2 | | 18E7 | --GPKATLQDIVLHLLEPQN- | 81 |
| consensus | | 56E7 | MHGKVPTLQDVVLELTPQTEI | 82 |
| regions | | 59E7 | MHGPKATLSDIVLDL------ | 83 |
| | 1-21 | Consensus | MHG***TL*D*VL*L*****- | 84 |
| | | 16E7 | ----------YSYEQLNDSSEEE---- | 85 |
| | | 31E7 | --LQPKATDLHSYEQLP---------- | 86 |
| | | 33E7 | LDLYPEPTDLYSYEQLSDSSDEDEGLD | 87 |
| | | 35E7 | --LEPEATDLYSYEQLSDS-------- | 88 |
| | 13-39 | Consensus | --L*P**TDL*SYEQL*DSS*E*---- | 89 |
| 128-3 | | 11E7 | MHGRLVTLKDIVLDLQPPD---- | 90 |
| | | 18E7 | MHGPKATLQDIVLHLEPQN---- | 91 |
| | | 45E7 | MHGPQATLQEIVLHLEPQN---- | 92 |
| | | 56E7 | MHGKVPTLQDVVLELTPQTEIDL | 93 |
| | | 59E7 | MHGPKATLSDIVLDL-------- | 94 |
| | | 70E7 | ------TLQEIVLDLYPYN---- | 95 |
| | 1-23 | Consensus | MHG***TL***VL*L*P**---- | 96 |
| 167-5 | | 11E7 | MHGRLVTLKDIVLDLQPPD---- | 97 |
| | | 18E7 | --GPKATLQDIVLHLEPQN---- | 98 |
| | | 56E7 | MHGKVPTLQDVVLELTPQTEIDL | 99 |
| | | 59E7 | MHGPKATLSDIVLDL-------- | 100 |
| | 1-23 | Consensus | MHG***TL*D*VL*L*P**---- | 101 |

^{#}Conformation epitope
^{$}Weak signal, possibly a conformation epitope

The E7 proteins of the different HPV strains have about 98 to about 106 amino acids. The amino acid positions as provided in Table 1 refer to the consensus sequence as published by Ohlenschlager et al., Oncogene (2006), 5953-5959.

It is an important aspect of the present invention that by using several different monoclonal antibodies as capture antigens it is possible to selectively bind the antigen to be identified, namely the HPV protein E7 from different high-risk strains of HPV since the combination of the different rabbit monoclonal antibodies covers all potential epitopes occurring in the high-risk strains of HPV.

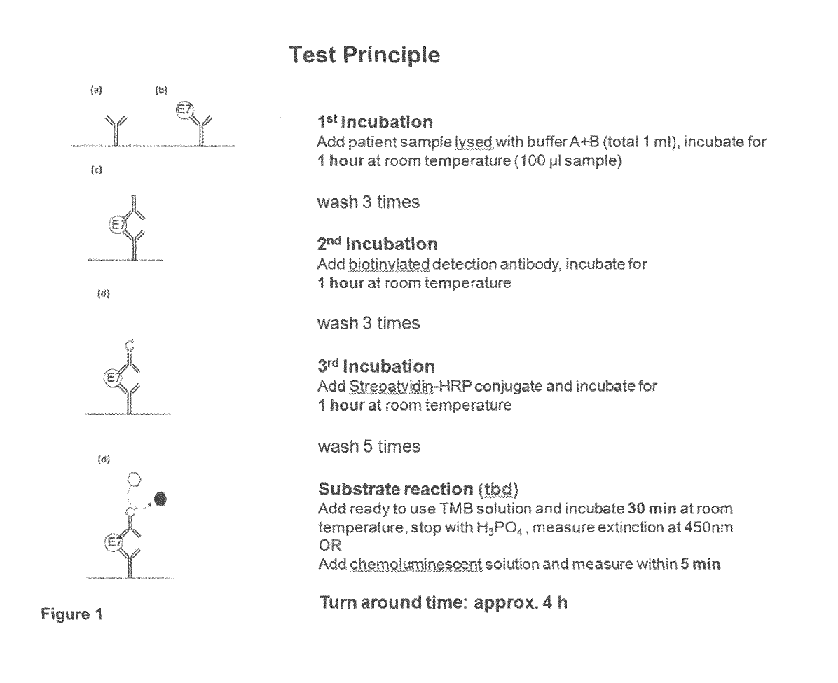

The test principle of a preferred embodiment is shown in FIG. 1. For performing a diagnostic test according to the invention a suitable test kit is prepared. At the surface of the wells of the reaction holes the capture antibodies are attached to each well of the titer plate. Then a biological sample obtained from a patient is pipetted into the well. The sample is usually lysed with a special lysis buffer and incubated for a sufficient time, preferably one hour, at room temperature. This allows the binding of potential E7 antigen to the capture antibody. Subsequently the wells are washed several times, preferably three times.

In the third step the wells are incubated with the detection antibody and the reaction mixture is incubated for a sufficient time, preferably around one hour at room temperature. In order to purify the well from unbound material the well is washed preferably three to six times with a washing buffer.

In the next step the signal producing means is linked to the detection antibody. This can preferably be done by a streptavidin-biotin binding. Then the wells are washed several times in order to avoid any unspecific reaction.

Finally the signal is created usually by adding a colourless substrate which is converted by the action of the signal performing means (enzyme) into a coloured product. For example a TMB solution can be used for the development of the colour. After a certain time, usually about 30 minutes, the reaction is stopped by addition of a stopping agent (e.g. H₃PO₄) and the extinction is measured at a suitable wavelength, preferably at about 450 nm.

Preferred embodiments of the present invention are described in more detail in the examples and the figures.

### Example 1 (Several Capture Antibodies in One Well for Detection of 12 hr Types Simultaneously)

In order to test the efficacy of different combinations of the rabbit monoclonal antibodies 75-3, 58-3, 84-2, 143-7, 159-1, and 146-8 different combinations of monoclonal antibodies were used as capture antibodies. The designation of the rabbit monoclonal antibodies correlates with the epitopes to which such antibodies bind as shown in Table 2.

TABLE 2
Capture Antibodies Present in Different Wells

| RabMab | RabMab | RabMab | RabMab | RabMab | RabMab |
|---|---|---|---|---|---|
| clone | clone | clone | clone | clone | clone |
| combination 1 | combination 2 | combination 3 | combination 4 | combination 5 | combination 6 |
| 75-12 | 143-7 | 143-7 | 146-8 | 75-12 | 143-7 |
| 58-3 | 58-3 | 58-3 | 159-1 | 58-3 | 58-3 |
| 84-2 | 84-2 | 159-1 | — | 84-2 | 84-2 |
| — | 159-1 | — | — | 146-8 | 146-8 |
| — | — | — | — | 159-1 | 159-1 |

Afterwards, purified recombinant E7 proteins of 14 different HPV types (2 low-risk types as negative control and 12 hr types), produced in E. coli, were added to the plate in order to determine the signal pattern of each RabMab combination. Additionally, buffer without any E7 protein served as a blank (negative) control.

As detection antibodies three different polyclonal goat antibodies (short goat 1-3) were used as mixtures (goat 1+2 for combination 1 to 5, Goat 1+2+3 for combination 6). For production of the detection antibodies, different E7 proteins or combinations thereof were used as immunogen with 16E7 for goat 1, 18E7 for goat 2, and a 1:1:1:1 mixture of the E7 proteins of HPV types 39, 51, 56, and 59E7 for goat 3. The goat antibodies were biotinylated to obtain best possible sensitivity.

The detection sera, shortly goats, are summarized in Table 3.

TABLE 3
Detection Antibodies

| | final | total | |
|---|---|---|---|
| | concentration in | concentration of | |
| Goats | assay | mixture of goat | Source |
| Goat 1 | 0.4-1.6 μg/ml | 1.2-4.8 μm/ml | Plasma |
| Goat 2 | 0.4-1.6 μg/ml | | Plasma |
| Goat 3 | 0.4-1.6 μg/ml | | Serum |

In the test recombinant E7 proteins obtained from the strains 6, 11, 16, 18, 31, 33, 35, 39, 45, 51, 52, 56, 58, and 59 were used. The E7 proteins were produced recombinantly.

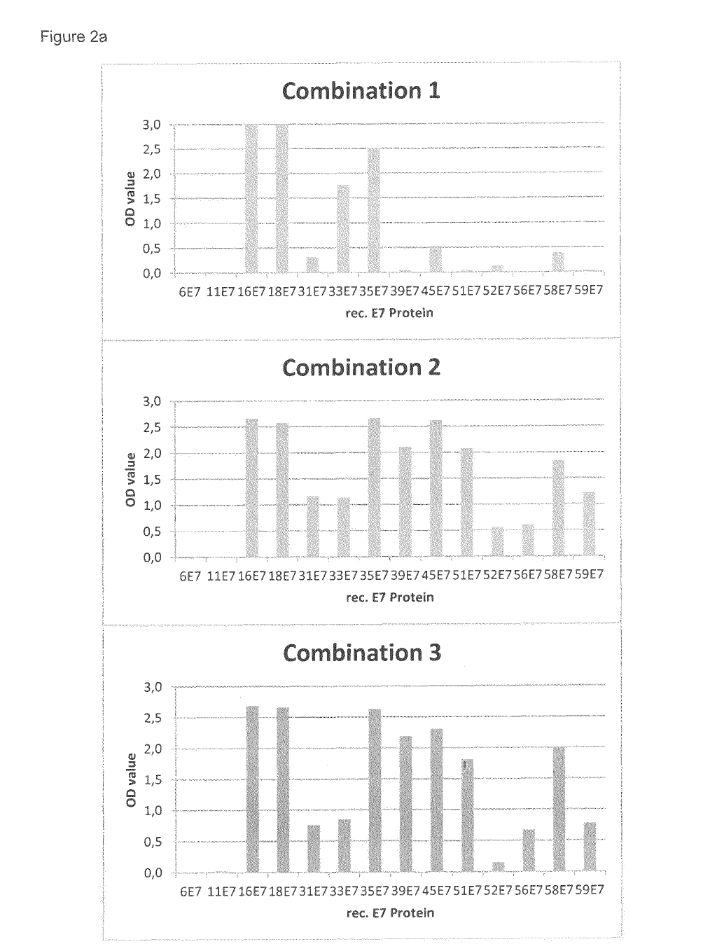

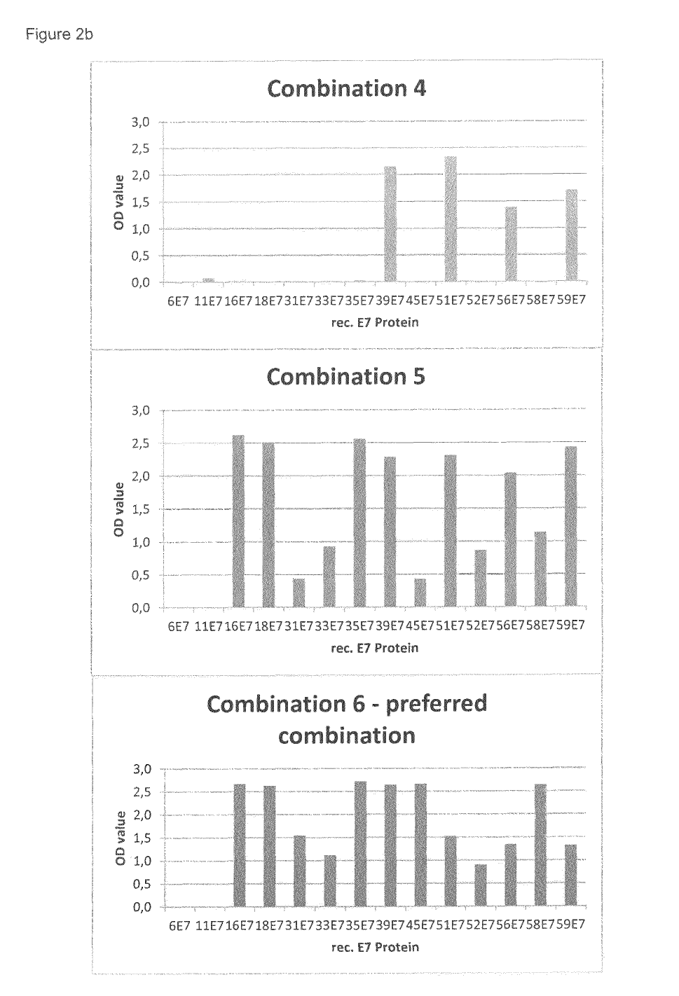

The results of the experiments can be seen in FIGS. 2 a)-b). The most preferred combination for detection of 12 high-risk HPV types of rabbit monoclonal antibodies is combination 6.

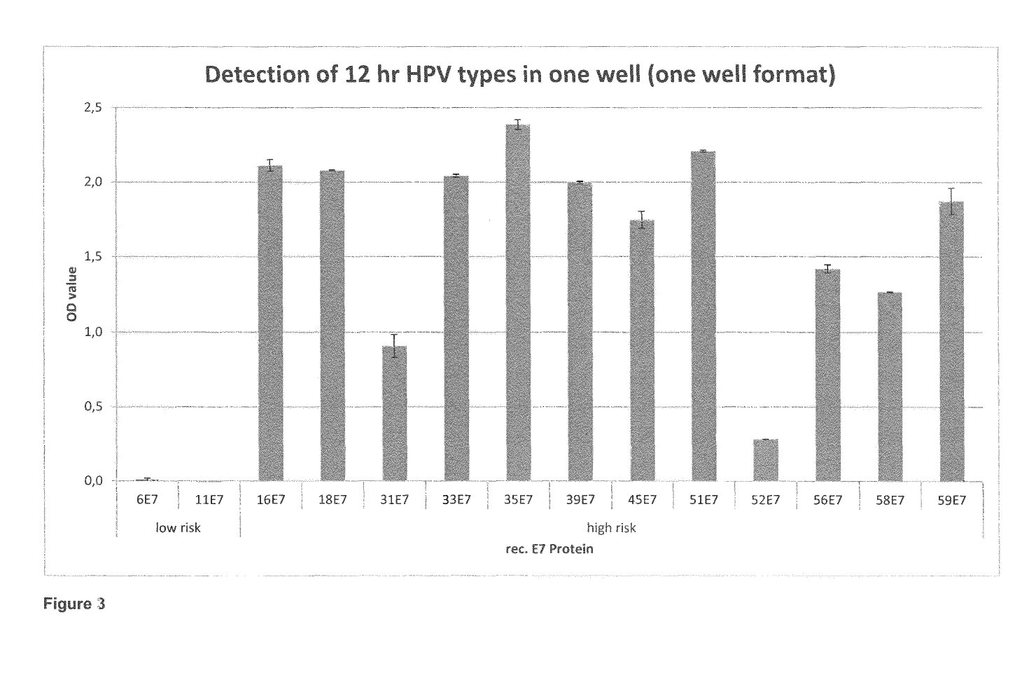

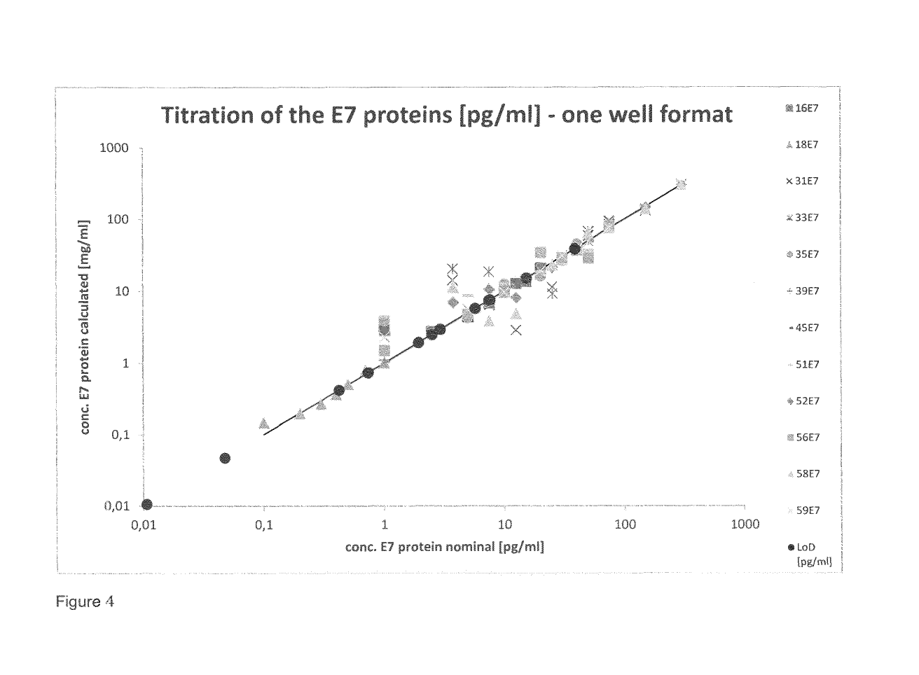

In an attempt to detect all E7 proteins in a single detection step, wells were coated with a mixture of rabbit monoclonal antibodies 143-7, 58-3, 84-2, 146-8 and 159-1. Detection was performed with a 1:1:1 mixture of biotinylated goat polyclonal antibodies goat 1, goat 2, and goat 3. With this setting the E7 oncoproteins of all high-risk HPV types analysed were detected, whereas signals obtained with low-risk HPV types 6 and 11 were in the background level. In this pan-high-risk E7 ELISA assay the detection limit varied between 0.1 picogram and 40 picogram (FIGS. 3 and 4) as shown with serial dilutions of all 12 hr HPV types.

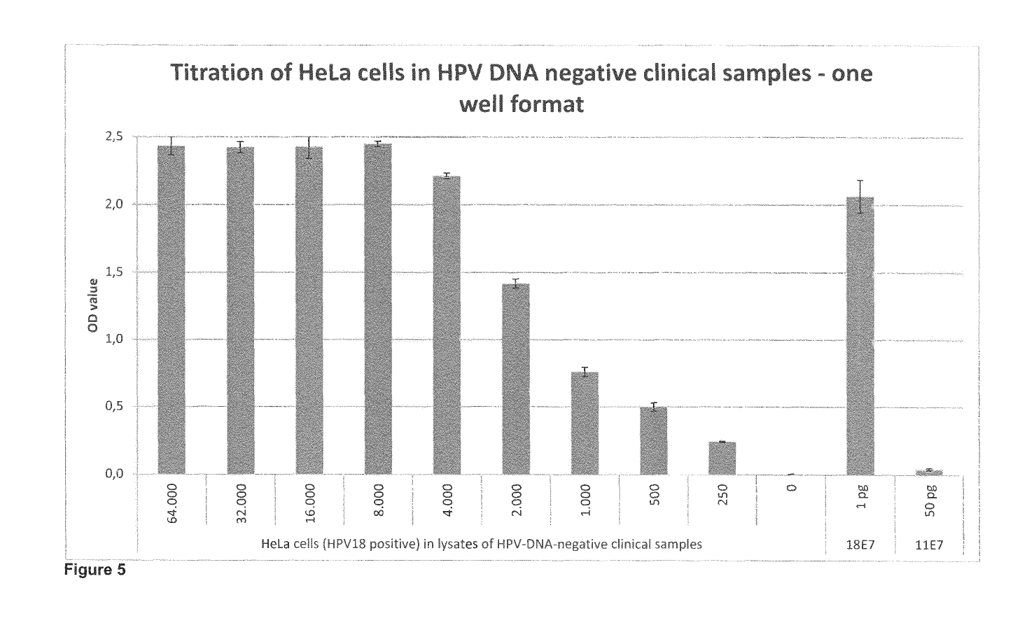

Control experiments (FIG. 5) revealed signals clearly over background with 250 HeLa cells (HPV-18 positive) spiked in HPV DNA negative cervical samples from patients without clinical findings. These summarized data suggest that sufficient sensitivity is given for this format to detect 12 high-risk HPV types in one well.

### Example 2 (One Sample in Several Reaction Wells)

Rabbit monoclonal antibodies 42-3, 143-7, 58-3, 80-2, 84-2, 128-3, and 146-8 were raised against combinations of hrE7 proteins and characterized for binding specificity by direct ELISA and epitope mapping. Different combinations of rabbit monoclonal antibodies directed against various E7 proteins were used as coating antibodies on standard 96-well plates. Subsequently, purified recombinant E7 proteins of different HPV types (produced in E. coli) were added to the coated plates and used as standards for the detection sensitivity of each particular combination of RabMabs.

Bound E7 proteins were detected by the addition of affinity purified goat antibodies (referred to as Goat1, Goat2 and Goat3, respectively) raised against different E7 proteins or combinations therefore which were used as immunogens, as follows:

In all sandwich ELISA tests the signals obtained without the addition of E7 (buffer) of with the addition of low-risk E7 protein (6E7 and 11 E7) was used as negative controls.

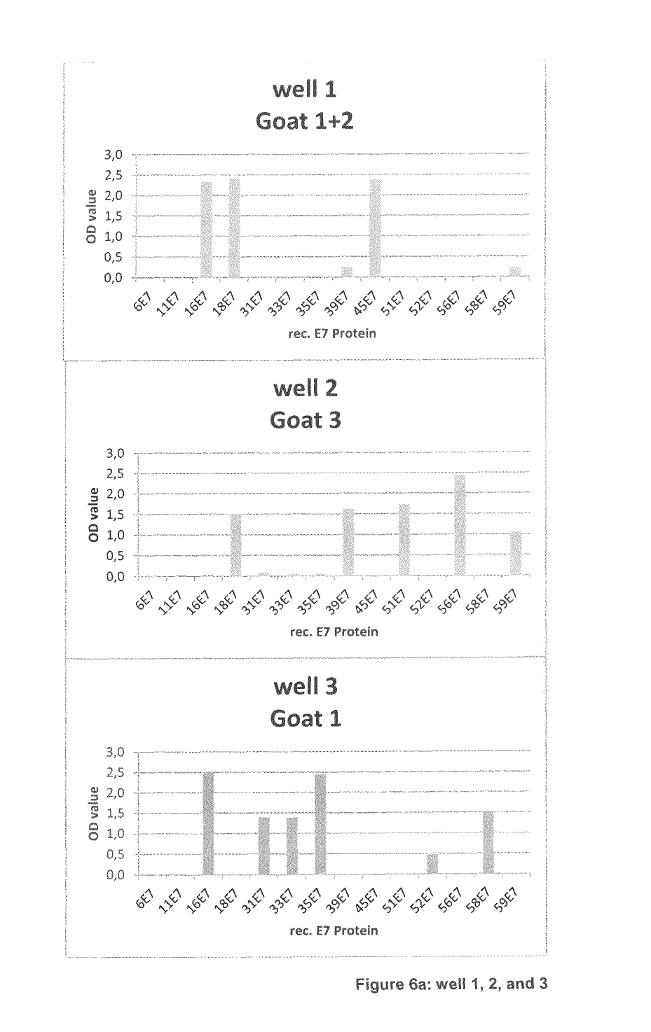

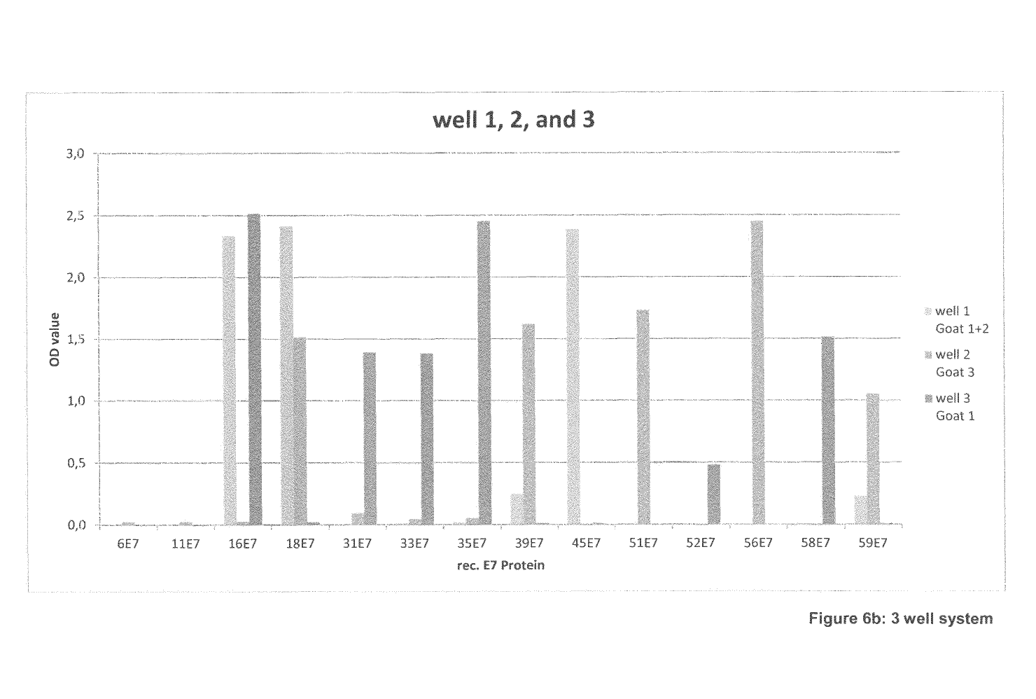

It was found that the combination (1:1) of RabMab 42-3 and 143-7 as capture antibodies, in combination with biotinylated polyclonal goat antibody goat 1 was capable to detect specifically the E7 proteins of HPV 16, HPV 18 and HPV 45 (FIGS. 6a and b).

For the simultaneous detection of the E7 proteins of HPV type 39, 51, 56 and 59, coating was performed with a combination (1:1) of rabbit monoclonal antibodies 128-3 and 146-8. Here biotinylated antibodies Goat 3 were used. With this combination of polyclonal and monoclonal antibodies the E7 proteins of HPV types 39, 51, 56 and 59 were detected (FIGS. 6a and b).

For the detection of the E7 proteins encoded by HPV types 31, 33, 35, 52, and 58, a combination of rabbit monoclonal antibodies 57-4, 80-2 and 84-2 were used for coating. For detection biotinylated antibody Goat 1 was used. Under these conditions, we were able to detect the E7 proteins of HPV 31, 33, 35, 52 and 58 (FIGS. 6a and b).

The format of well 1 (coating antibody RabMab 42-3 and RabMab 143-7; detection with a 1:1 mixture of biotinylated goat antibody goat 1 and goat 2, see above, FIGS. 6a and b) was used to determine the amount of E7 protein present in cervical cancer cell lines and in cervical smears derived from patients.

### Example 3 (Control Experiments)

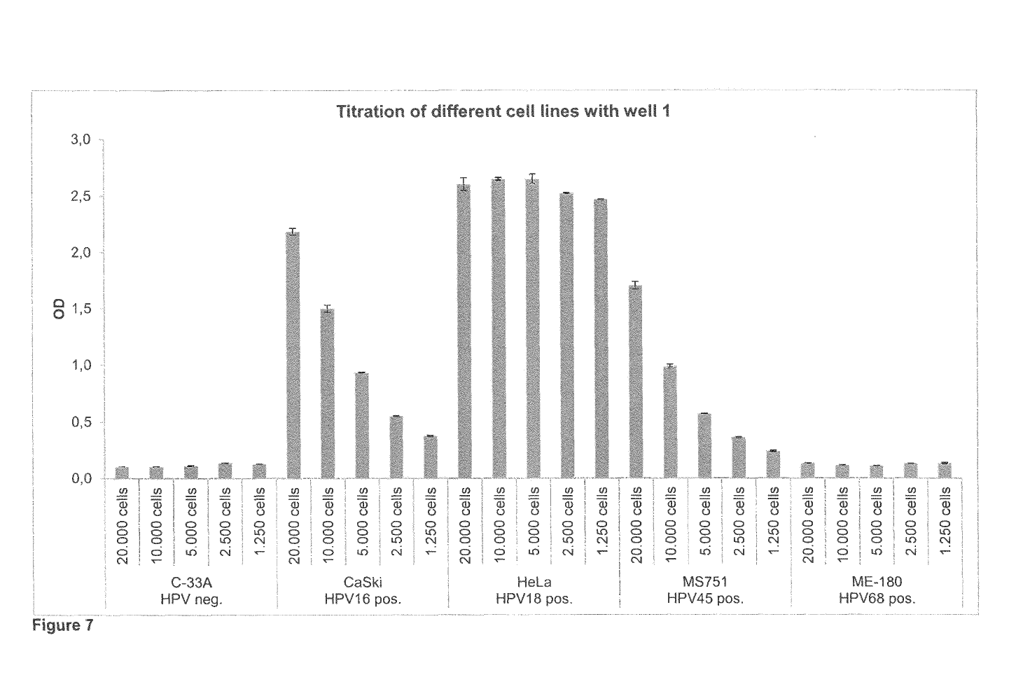

Control experiments with well 1 (FIG. 7) revealed signals clearly over background with 250 HeLa cells (HPV-18 positive) or 1200 Caski cells (HPV-16 positive) or 1250 MS751 cells (HPV-45 positive). No signals were detected for the negative control cervical cancer cell lines C33a (HPV negative) as well as the HPV-68 DNA positive cell line ME-180.

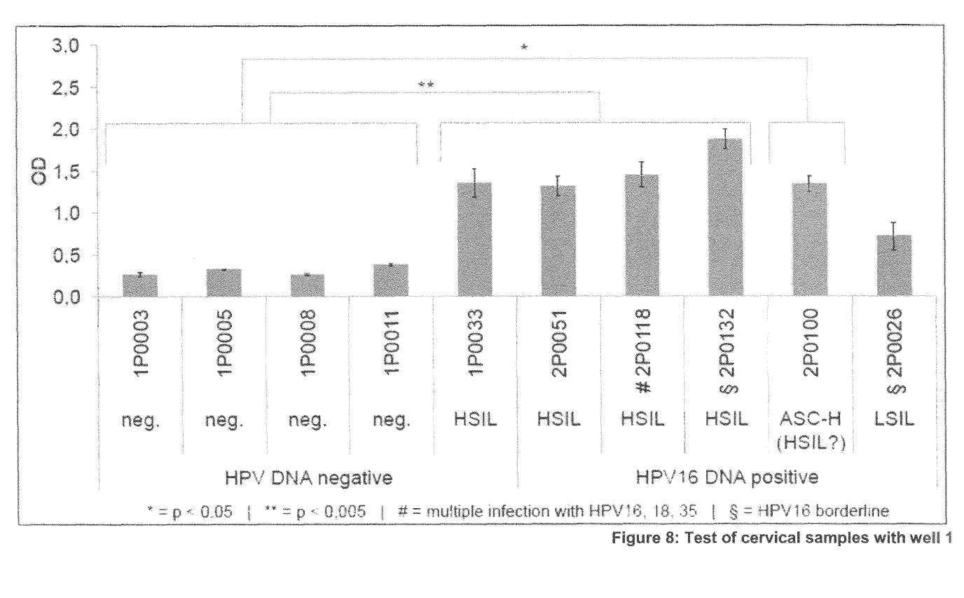

The E7 signal in HPV DNA negative samples without clinical findings was in the range of the background signal, whereas the HVP DNA positive, clinically abnormal samples, clearly displayed E7 content above background (FIG. 8). These samples were characterized as CIN2 and/or above and showed high grade lesions, confirmed by histology. Furthermore, for one clinical sample which was HPV DNA negative tested but clinical abnormal with a high grade lesion, a strong positive signal for E7 was detectable. These data suggest that the setting of ELISA well 1 is already in the correct dynamic range to detect clinically abnormal smears.

### Example 4

The particularly preferred rabbit monoclonal antibodies 42-3, 143-7, 58-3, 80-2, 84-2, 128-3, and 146-8, which are used for the three well system described in Example 2 (one sample in several reaction wells), were coated together (1:1:1:1:1:1:1) in one well of standard 96-well plates. Subsequently, purified recombinant E7 proteins of 12 different hrHPV types (produced in E. coli) were added to the coated plates. Detection of bound E7 proteins was performed with a mixture of biotinylated goat polyclonal antibodies goat 1, goat 2, and goat 3 (as described in Example 2).

In order to compare the performance of the test according to the present invention with the test method as described in the prior art, different combinations of monoclonal antibodies were compared.

In well 1 (Table 4 and Table 5) the antibodies 42-3 and 143-7 as described in US 2013/0029322 were used. Furthermore, two other different combinations of monoclonal antibodies (not disclosed in prior art) were used. In addition, all monoclonal antibodies used in Example 2 were combined in one well. The arrangement of the monoclonal antibodies is shown in Table 4. For detecting the results polyclonal goat antisera was used as described in more detail in Table 5.

TABLE 4

| | | | RabMab- | | | |
|---|---|---|---|---|---|---|
| | | | clone | | final | |
| RabMab- | RabMab- | RabMab- | well | final | concentration | |
| clone | clone | clone | 1 + 2 + 3 in | concentration | of RabMab | |
| well 1 | well 2 | well 3 | one well | in assay | mixture | source |
| 42-3 | 128-3 | 58-3 | 42-3 | 0.4-2 μg/ml | 2.8-10 μg/ml | Hybridom |
| 143-7 | 146-8 | 80-2 | 143-7 | 0.4-2 μg/ml | | Hybridom |
| — | — | 84-2 | 128-3 | 0.4-2 μg/ml | | Hybridom |
| — | — | — | 146-8 | 0.4-2 μg/ml | | Hybridom |
| — | — | — | 58-3 | 0.4-2 μg/ml | | Hybridom |
| — | — | — | 80-2 | 0.4-2 μg/ml | | Hybridom |
| — | — | — | 84-2 | 0.4-2 μg/ml | | Hybridom |

TABLE 5

| | final concentration | total concentration of | |
|---|---|---|---|
| goats | in assay | goat mixture | source |
| goat 1 | 0.4-1.6 μg/ml | 1.2-4.8 μg/ml | plasma |
| goat 2 | 0.4-1.6 μg/ml | | plasma |
| goat 3 | 0.4-1.6 μg/ml | | serum |

The setting of table 4 was tested with recombinant proteins of 12 high risk HPV types: (i) the three well system as described in example 2 (one sample in several reaction wells) with well 1 [antibodies 42-3 and 143-7 as described in US 2013/0029322], well 2 [antibodies 128-3 and 146-8] and well 3 [antibodies 58-3, 80-2, and 84-2] (both antibody combinations not disclosed in prior art), as well as (ii) all antibodies of example 2 combined in one well [antibodies 42-3, 143-7, 128-3, 146-8, 58-3, 80-2, and 84-2].

The (iii) preferred combination from example 1 (several capture antibodies in one well for detection of 12 hr types simultaneously) was tested in addition.

The results are shown in table 6: Whereas a detection of 12/12 hrHPV E7 proteins distributed over the three different wells was possible (Example 2 and column 1-3 with 4/12 hrHPV E7 proteins for well 1 [33.3%], 5/12 hr HPV E7 proteins for well 2 [41.7%], and 6/12 hrHPV E7 proteins for well 3 [50%], only 8/12 hr E7 proteins (66.7%; 16E7, 18E7, 35E7, 39E7, 45E7, 51E7, 56E7, and 59E7) were detected when the RabMabs used in the three well system of Example 2 were combined in one well (column 4). 31E7, 33E7, 52E7, and 58E7 were not detectable at all with this RabMab combination.

In the contrary, the combination used in Example 1 (several capture antibodies in one well for detection of 12 hr types simultaneously) resulted in detection of 12/12 hrHPV E7 proteins in one well (100%, column 5).

The results of this experiment can be summarized in Table 6.

TABLE 6

| | prior art | | | | preferred |
|---|---|---|---|---|---|
| Target (HPV | Well 1 | | | Well 1 + 2 + 3 | one well |
| Subtype - E7 | Goat | Well 2 | Well 3 | in one well | format Goat |
| Protein | 1 + 2 | Goat 3 | Goat 1 | Goat 1 + 2 + 3 | 1 + 2 + 3 |
| 16E7 | + | − | + | + | + |
| 18E7 | + | + | − | + | + |
| 31E7 | − | − | + | − | + |
| 33E7 | − | − | + | − | + |
| 35E7 | − | − | + | + | + |
| 39E7 | − | + | − | + | + |
| 45E7 | + | − | − | + | + |
| 51E7 | − | + | − | + | + |
| 52E7 | − | − | + | − | + |
| 56E7 | + | + | − | + | + |
| 58E7 | − | − | + | − | + |
| 59E7 | − | + | − | + | + |
| detected/ | 4/12 | 5/12 | 6/12 | 8/12 | 12/12 |
| total | | | | | |
| detection | 33.3% | 41.7% | 50.0% | 66.7% | 100.0% |
| [%] | | | | | |

1. A diagnostic test method for the detection of an E7 protein of a human papilloma virus in a biological sample, said method comprising the steps of:
(a) providing a sandwich enzyme linked immunosorbent assay (ELISA) that includes (i) a capture antibody comprising at least three different rabbit monoclonal antibodies that bind to at least three different epitopes and (ii) a detection antibody comprising at least two different polyclonal anti E7 antibodies obtained by immunization with different antigens; and
(b) detecting the E7 protein of a human papilloma virus in the biological sample using the ELISA of step (a).

2. The diagnostic test method according to claim 1, characterized in that the capture antibody comprises at least four rabbit monoclonal antibodies that bind to at least four different epitopes.

3. The diagnostic test method according to claim 1, characterized in that the capture antibody comprises at least five rabbit monoclonal antibodies that bind to at least five different epitopes.

4. The diagnostic test method according to claim 1, characterized in that the detection antibody comprises at least three different polyclonal anti E7 antibodies.

5. The diagnostic test method according to claim 1, characterized in that the detection antibody comprises at least one polyclonal antibody obtained by immunization of an animal with HPV-16 E7.

6. The diagnostic test method according to claim 1, characterized in that the detection antibody comprises at least one polyclonal antibody obtained by immunization of an animal with HPV-18 E7.

7. The diagnostic test method according to claim 1, characterized in that the detection antibody comprises at least one polyclonal antibody obtained by immunization of an animal with a mixture of the E7 proteins of HPV types 39, 51, 56 and 59.

8. The diagnostic test method according to claim 1, characterized in that the rabbit monoclonal antibodies of the capture antibody are selected from the group consisting of antibodies that bind to epitopes located within the following stretches of amino acids: 1-17; 11-37; 69-85; 31-51; 15-31; 85-98.

9. The diagnostic test method according to claim 1, characterized in that the same biological sample is tested in several reaction wells, whereby each reaction well comprises at least one rabbit monoclonal antibody and the rabbit monoclonal antibodies adhered to each of said wells is different from the antibody in the other well and that each of the wells used for one sample is reacted with at least one polyclonal anti E7 antibody whereby the polyclonal anti E7 antibodies that are reacted with said biological sample are different for each well.

10. The diagnostic test method according to claim 1, characterized in that in one test well of the ELISA, the capture antibody comprises at least five different rabbit monoclonal antibodies that bind to at least five different epitopes and the detection antibody comprises at least three different polyclonal anti E7 antibodies.

11. The diagnostic test method according to claim 1, characterized in that the biological sample is obtained from a patient suffering from cervical cancer.

12. The diagnostic test method according to claim 1, characterized in that the biological sample is obtained from a patient suffering from head and neck cancer.

13. The diagnostic test method according to claim 1, characterized in that the biological sample is obtained from a patient suffering from anal carcinoma.

14. A diagnostic test kit for performing an immunological test method according to claim 1, wherein said kit comprises (a) means for performing an ELISA sandwich test, (b) means for the preparation of washing solutions, (c) polyclonal detecting antibodies and (d) means for the development of a signal.

15. The diagnostic test kit according to claim 14, wherein said means for performing an ELBA sandwich test includes a solid phase coated with monoclonal rabbit capture antibodies.
